(19) United States
(12) Patent Application Publication
(10) Pub. No.: US 2021/0177699 A1
(43) Pub. Date: Jun. 17, 2021

(54) Injection Port For Connecting With A Medical Fluid Container And Methods For The Production Of Same

(71) Applicant: Fresenius Medical Care Holdings, Inc., Waltham, MA (US)

(72) Inventors: Terry B. KEHNE, Knoxville, TN (US); Donald L. KING, JR., Knoxville, TN (US)

(73) Assignee: Fresenius Medical Care Holdings, Inc., Waltham, MA (US)

(21) Appl. No.: 17/109,173

(22) Filed: Dec. 2, 2020

Related U.S. Application Data
(60) Provisional application No. 62/946,987, filed on Dec. 12, 2019.

(51) Int. Cl. A61J 1/14; B29C 45/00

(52) U.S. Cl. CPC A61J 1/1475; A61J 1/1406; A61J 1/10; B29C 45/0062; A61J 1/1431

(57) ABSTRACT

The present invention relates to an injection port for connecting with a container, such as a medical fluid container. The injection port includes a barrel, a septum, and a cap, which are sequentially formed via a three-shot injection molding process. The present invention further relates to an assembly that includes the injection port connected to a container. Further, the present invention relates to a method of making the injection port utilizing a three-shot injection molding process.

This application claims the benefit under 35 U.S.C. § 119(e) of prior U.S. Provisional Patent Application No. 62/946,987, filed Dec. 12, 2019, which is incorporated in its entirety by reference herein.

### BACKGROUND OF THE INVENTION

The present invention relates to a port and closure assembly that can be used to access a container that can contain a liquid such as a liquid for a medical use. The present invention more specifically relates to an injection port, such as one for connecting with a medical fluid container (e.g., an infusion fluid container) that can be useful in dialysis or other medical procedures, as well as methods for manufacturing the injection port.

Ports, commonly referred to as injection ports, are used in many medical procedures and can be of a tubular structure having an inner bore that extends from a base that is ultimately secured onto a container. This tubular structure is at times referred to as a barrel. Located within a part of the inner bore is a wall or barrier that can be pierced by a needle, cannula, or similar medical device. The wall or barrier is commonly referred in some fields as a septum, and provides a barrier to any fluid contained within the container. The septum also provides protection against contamination of the port and inner bore.

Some port assemblies commercially available are formed from two pieces, for instance, a septum and a barrel and other port assemblies are formed by three pieces, a septum, a barrel, and a cap located at least partially around the septum. While the current port assemblies available are useful for their designed purpose, there is always a demand for an improved design and/or improved method of making a port assembly. For instance, in some manufacturing processing, while molds are used to separately form one or two parts of the port assembly, there is often a further step required to assemble the parts together to form the finished piece. The assembly requires further time, cost, and potential contamination to the finished piece. Also, in at least several current port assembly designs, there are air spaces that are present once the parts of the port assembly are assembled together. With such air spaces, there is then the need for a sterilization procedure of the assembled part, which again takes time and adds additional cost and complexity to the manufacturing process.

Accordingly, a need exists to provide a port assembly, e.g., an injection port, and a manufacturing process, that overcome one or more of the disadvantages mentioned above.

### SUMMARY OF THE PRESENT INVENTION

A feature of the present invention is to provide an injection port or port assembly that requires no assembly after the molding process that forms the parts of the injection port.

A further feature of the present invention is to provide an injection port wherein there is an absence of any trapped air spaces (outside of the fluid path) in the injection port and an absence of any trapped air spaces in or between the pieces that form the injection port.

A further feature of the present invention is to provide an injection port that avoids the need for pre-sterilization after the injection port is formed.

An additional feature of the present invention is to provide a method of making an injection port wherein the method can form all parts of the injection port in a continuous or semi-continuous molding process.

Additional features and advantages of the present invention will be set-forth in part in the description that follows, and in part will be apparent from the description, or may be learned by practice of the present invention. The objectives and other advantages of the present invention will be realized by means of the elements and combinations particularly pointed out in the description and appended claims.

To achieve these and other advantages, and in accordance with the purposes of the present invention, as embodied and broadly described herein, the present invention relates to an injection port or port assembly, for connecting with a container. The injection port includes a barrel, a septum, and a cap. The barrel has a tubular bore for fluid communication with an interior of a container, such as a fluid container or medical fluid container, and an injection site end configured to hold the septum. The septum is in contact with the injection site end of the barrel. The septum provides a sealing partition to the injection site end of the barrel. The septum is configured to be resealable after being pierced by an injection needle or cannula. The cap has an annular shape and circumscribes the septum, holding, at least partly, the septum to the barrel at the injection site end. The cap has a central opening enabling access to the septum. The cap further circumscribes the injection site end of the barrel, wherein the cap, in part, secures the septum in place. The barrel, the septum, and the cap are sequentially formed via a three-shot injection molding process.

The present invention further relates to an assembly that includes the injection port of the present invention and a container, such as a fluid container or medical fluid container. The fluid container has an interior and the tubular bore of the barrel is in fluid communication with the interior of the fluid container.

The present invention also relates to a method of making the injection port of the present invention. The method includes a three-shot injection molding process (three shots), wherein, during a first shot of the three-shot injection molding process, the barrel is formed, during a second shot of the three-shot injection molding process, the septum is formed, and during the third shot of the three-shot injection molding process, the cap is formed.

It is to be understood that both the foregoing general description and the following detailed description are exemplary and explanatory only and are intended to provide a further explanation of the present invention, as claimed.

### BRIEF DESCRIPTION OF THE DRAWINGS

The drawings represent various design features of the injection port or port assembly. Similar referencing identifiers in different figures can refer to similar features unless indicated otherwise. The drawings are not necessarily to scale.

FIG. 1 is a diagram showing a prospective view of the overall injection port of the present invention and connected to a container, such as a fluid container and the option for a further conduit.

FIG. 2 is a diagram showing the cross-sectional view of the overall injection port of the present invention.

FIG. 3 is a diagram showing the cross-sectional view of the barrel of the injection port of the present invention.

FIG. 4 is a diagram showing the cross-sectional view of the septum of the injection port of the present invention.

FIG. 5 is a diagram showing the cross-sectional view of the cap of the injection port of the present invention.

FIGS. 6-8 are cross-sectional diagrams showing an example of the molds for the first shot, second shot, and third shot, respectively, for one exemplary multi-shot injection molding process.

FIG. 9 is a diagram showing an example of a molded barrel with apertures to receive material that forms the cap.

### DETAILED DESCRIPTION OF THE PRESENT INVENTION

The present invention relates to an injection port or a port assembly. In the description that follows, the term ‘injection port’ is used but it is to be understood that the injection port of the present invention can be referred to as a port assembly or as a port that can be connectable to or in fluid communication with a container, such as a fluid container or medical fluid container. The container can be an infusion container, such as an infusion bag, or can be a container that makes use of a port connection for any medical use or procedure, including, but not limited to, a dialysis procedure, an intravenous procedure, an ingestion procedure, a rinsing procedure, or the like. For instance, the fluid container can contain one or more components of a dialysis solution or fluid. There are no limitations on the container or fluid container that the injection port can be used with and any commercially available fluid containers, especially medical fluid containers, can be used with the injection port of the present invention.

In more detail and referring to the figures as an example of a design, the injection port 1 of the present invention can be connected to a medical fluid container 2. The injection port can comprise, consist essentially of, consist of, or include a) a barrel 3, b) a septum 5, and c) a cap 7.

The barrel 3 of the injection port 1 has a tubular bore 11 for fluid communication with an interior of the container, such as a medical fluid container 2. The barrel has an injection side end 51 and an opposite end 52. The opposite end 52 can at least partially be used to connect the injection port 1 to an opening of a medical fluid container 2.

The injection site end is configured to hold the septum 5. The septum is in contact with the injection site end 51 of the barrel. The septum provides a sealing partition 17 to the injection site end of the barrel. The septum is configured to be resealable after being pierced by an injection needle or cannula.

The cap 7 of the injection port 1 is a cap having an annular shape. The cap 7 circumscribes the septum 5, holding, at least partially, the septum 5 to the barrel 3 at the injection site end 51. The cap 7 has or includes a central opening 49 enabling access to the septum 5. The cap 7 further circumscribes the injection site end 51 of the barrel, wherein the cap 7, in part, secures the septum 5 in place.

Preferably, the barrel, the septum, and the cap are sequentially formed via a three-shot injection molding process, which is described in more detail hereinafter with respect to preferred embodiments.

The barrel 3 can comprise, consist essentially of, consist of, or include, at least one thermoplastic material, e.g., at least one thermoplastic polymer. The thermoplastic material can be or include one or more thermoplastics such as, but not limited to, a polymer that is polycarbonate, polystyrene, polypropylene, polyethylene, and/or acrylonitrate butadiene, or a copolymer of or having one or more of these polymers, and the like. The thermoplastic can be a homopolymer or copolymer. The thermoplastic can be considered a rigid thermoplastic. A rigid thermoplastic can be a thermoplastic with a hardness as measured by Rockwell R Scale-ASTM D785 at 73 deg F. of about 40 or higher, such as 50 or higher, or 60 or higher, such as from about 40 to 121. In the case of polycarbonate, the hardness can be, as an example from about 65 to about 121. In the case of polystyrene, the hardness can be from about 70 to 115. In the case of polypropylene, the hardness can be from about 80 to about 90. In the case of polyethylene, the hardness can be from about 40 to about 70. In the case of acrylonitrate butadiene, the hardness can be from about 80 to about 115. For instance, the polyethylene can be a high density PE or ultra high molecular weight PE. The material used to form the barrel can be a blend of thermoplastic materials. The thermoplastic(s) are all commercially available. Commercially available sources include, but are not limited to, Dow and Exxon Mobil (e.g. Polypropylene PP 1013 H1). The material used to form the barrel can contain, in addition to the thermoplastic material, one or more additives. Examples of such additives, include, but are not limited to, fillers, nanobarcodes, RFIDs, electrically conductive particles, thermally conductive particles, anti-microbial agent(s), and/or colorant(s), and the like. The one or more additives, if present, can be present in any amount, such as from 0.01 wt % to 5 wt % or more based on the total weight of the material forming the barrel.

Generally, the material used to form the barrel contains at least 90 wt % thermoplastic polymer(s), such as from 90 wt % to 100 wt %, or 90 wt % to 99 wt %, or 90 wt % to 95 wt % thermoplastic polymer(s) based on the total weight of the material used to form the barrel.

The barrel 3 has a base 59 and the base has an inner diameter 61. The injection site end (or upper end) 51 has an inner diameter 63 and an inner sidewall 27. The inner diameter 61 of the base 59 can be smaller than the inner diameter 63 of the injection site end 51 of the barrel 3.

Typical dimensions of the barrel are conventional. Exemplary dimensions are a bottom or lower outer diameter 69 of from 4 mm to 10 mm, such as about 6 to 8 mm and an inner diameter 61 of from 2 mm to 6 mm, such as about 3.5 to 4.5 mm. The upper or injection side end 51 can have an outer diameter 65 of from 8 mm to 12 mm, such as 9 mm to 10 mm, and an inner diameter 63 of from 6 mm to 10 mm, such as from 7 mm to 9 mm. The length 52 of the lower part or base 59 of the barrel 3 can be from 12 mm to 20 mm, such as from about 14 mm to 18 mm, and the length 53 of the upper part of the barrel (at the injection site end 51) can be from 4 mm to 8 mm, such as from 5 mm to 7.5 mm.

The shape of the barrel can be such that the bottom end or base 59 has an outer sidewall surface 13 that is parallel to an inner sidewall surface 15 (fluid contacting side) or the outer sidewall surface can have an angled design such that the outer sidewall angles away from the inner sidewall by 0.1 to 5 degrees, such as from 0.2 to 2 degrees. This can be considered a draft angle on the outer diameter side of the lower part of the barrel 52. When this occurs, the outer diameter of the bottom end of the barrel has an outer diameter that is different from the outer diameter at one or more regions of the barrel located above this point, closer to the injection site end. This change in outer diameter can be gradual. The base 59 can have an outer diameter that can be from 1% to 10% smaller than the outer diameter at the upper point of the bottom end. The inner diameter 61, lower portion of the barrel can optionally have a draft angle that is the same or similar to the draft angle for the outer diameter of the barrel described above as an option. If this occurs, the inner diameter 61 at the lower opening 59 can be different, such as from 1% to 10% larger or smaller than the inner diameter at the top of the lower barrel 52.

The base 59 can include a connecting end that can be used to connect the injection port to a connecting end 71 of a container or fluid container, such as a medical fluid container, such that the tubular bore 11 of the barrel 3 is in fluid communication with an interior of the container.

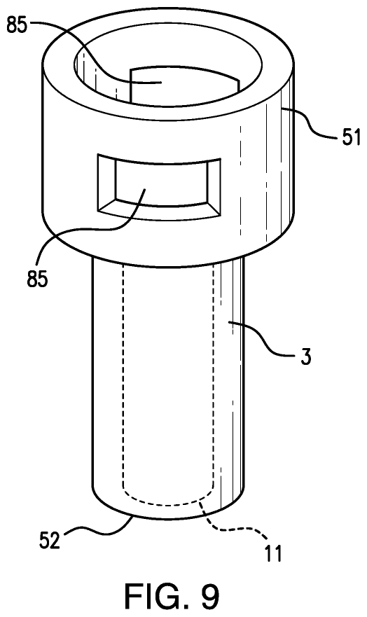

The barrel 3 can comprise or include one or more outwardly protruding ridges 21, at least at the injection site end 51. The ridge (or ridges) can be present and designed as part of the shape of the barrel 3 by the mold design used to form the shape of the barrel. The ridge or ridges can circumscribe an outside surface of the barrel at the injection site end (or upper end) 51. As discussed in more detail herein, the cap 7 is designed to have a complimentary notch, indent, or receiving groove 43 configured to engage the ridge 21. Although the ridge is not required in the design, the ridge can further assist in securing the cap 7 to the barrel 3. The ridge can have any design or shape, for instance, a triangular cross-section, a half-circle cross-section, a barb-shaped cross-section, or the like. As an option, the ridge, instead of being located on the outside of the barrel, can instead be located on the inside of the cap, and a complimentary notch, indent, or receiving groove can be located on the outside of the barrel and configured to engage the ridge on the cap. One or more ridges or protrusions can be used and the term “ridges” as used herein encompasses one or more ridges. As an option, with an example shown in FIG. 9, the surface of the barrel at the injection site end (or upper end) 51 can have one or more aperture 85 (in FIG. 9) so that upon the injection of the material forming the cap, a portion of that material fills the aperture so as to form a locking joint or type of pin with the surface of the barrel 51. For instance, if two apertures are used, the apertures can be 180 deg from each other on the outside surface of the barrel side 51. The aperture can have any shape, such as circular, triangular or other geometric shape. The size of the aperture can be from about 0.01 mm to 0.1 mm in cross-sectional area, as an example or can be other sizes. The aperture(s), for instance, can be located in a location that is the same or similar to the location of ridge 21. The aperture(s) configuration can permit pressure to be exerted on the septum. This pressure can contribute or provide a ‘self-sealing’ feature to the septum once an injection needle or cannula has been removed from the septum. This feature can impart a pre-load to the septum and improve the sealing ability of the septum after a needle or cannula is removed from the septum.

One or more ridges 75 can also be utilized to assist in securing the base or bottom surface 35 of the septum 5 to the upper end 51 of the barrel 3 (at the injection site end), and this location is also referred to as the inner shoulder 25 of the barrel 3. Thus, these one or more ridges can be part of the barrel design and the barrel can comprise or include one or more ridges at least at the inner shoulder 25 that engages the base or bottom end 35 of the septum. The ridge (or ridges) can be present and designed as part of the shape of the barrel by the mold design used to form the shape of the barrel. The ridge or ridges circumscribe an upper surface of inner shoulder 25 of the barrel at a point adjacent to where the inner bore of the barrel meets the base or bottom surface 35 of the septum. As discussed in more detail, the septum 5 can be designed to have a complimentary notch, indent, or receiving groove 37 configured to engage the ridge 75. This ridge is not required in the design. The ridge(s) can further assist in securing the septum to the barrel. The ridge(s) can have any design or shape, for instance, a triangular cross-section, a half-circle cross-section, a barb-shaped cross-section, or the like. As an option, the ridge, instead of being located on the barrel, can instead be located on the septum, and a complimentary notch, indent, or receiving groove can be located on the barrel and configured to engage the ridge on the septum. Again, one or more ridges can be used and the term “ridge” as used herein encompasses one or more ridges. The ridge(s) configuration can permit pressure to be exerted on the septum. This pressure can contribute or provide a ‘self-sealing’ feature to the septum once an injection needle or cannula has been removed from the septum. This feature can impart a pre-load to the septum and improve the sealing ability of the septum after a needle or cannula is removed from the septum.

With respect to the septum 5, the septum has a diameter 81 and an outer sidewall 39. The septum is located within the injection site end 51 (or upper end) of the barrel 3 such that the outer sidewall 39 of the septum 5 is in contact with the inner sidewall 27 of the injection site end of the barrel. A top surface 33 of the septum can be flush with the top surface 29 of the barrel 3.

The septum has the same or about the same (±0.1 to 0.2 mm) outer diameter 81 as the inner diameter 63 of the injection site end of the barrel 3 as described herein. Preferably, the outer diameter of the septum is such that no air gaps or pockets exist between the septum and the inner sidewall or contacting surface of the injection site end of the barrel.

Further, with respect to the interaction between the barrel and the septum, the injection site end 51 of the barrel 3 defines the inner shoulder 25. The septum has the inner axial surface (or bottom surface) 35, the outer axial surface (or top surface) 33, and the outer sidewall 39, and a portion of the inner axial surface 35 of the septum 5 rests against the inner shoulder 25 of the barrel 3.

Generally, the thickness (or height) 89 of the septum is the same or about the same (±0.5 mm or ±0.15 mm) as the height of the inner sidewall 27 of the injection site end 51 of the barrel 3.

The septum can comprise, consist essentially of, consist of, or be made from one or more elastomeric or thermoelastic materials (e.g., a thermoplastic elastomer). Examples of such materials include, but are not limited to, styrene-ethylene-butadiene-styrene SEBS), thermoplastic olefin, thermoplastic vulcanizate, thermoplastic polyurethane, melt processible rubber, co-polyester-ether, polyether block amide, polyisoprene and the like. The thermoplastic elastomer can be a homopolymer or copolymer of any one or more of these thermoplastic elastomers. The thermoplastic elastomer can be a silicone or include a silicone. The material used to form the septum can contain, in addition to the thermoelastic material, one or more additives. Examples of such additives, include, but are not limited to, fillers, nanobarcodes, RFIDs, electrically conductive particles, thermally conductive particles, anti-microbial agent(s), colorant(s), and the like. The one or more additives, if present, can be present in any amount, such as from 0.01 wt % to 5 wt % or more based on the total weight of the material forming the septum.

Generally, the material used to form the septum contains at least 90 wt % elastic, elastomeric, or thermoelastic polymer(s), such as from 90 wt % to 100 wt %, or from 90 wt % to 99 wt %, or from 90 wt % to 95 wt %, based on the total weight of the material used to form the septum. The material used to form the septum can have a Shore A hardness of from about 30 to about 70, such as from 35 to 50 (based on the Shore A durometer scale following ASTM D2240 type A).

With regard to the cap 7, the cap assists in securing the septum 5 to the barrel 3, or vice versa. The cap 7 can have an annular shape that circumscribes the septum 5. The cap includes a central opening 49 enabling access to the septum. The cap further circumscribes the injection site end 51 of the barrel 3.

The cap 7 has an outer diameter 91 of from 9 mm to 15 mm, such as from 10 mm to 12 mm at the contact point of where the inner sidewall 93 of the cap 7 contacts the outer sidewall 31 of the injection site end 51 of the barrel 3. The cap 7 has an inner diameter 95 that is the same or about the same (±0.15 mm) as the outer diameter of the upper injection end 65 of the barrel. The diameter of the bottom opening 57 that defines the annular shape of the cap can be the same as the inner diameter 95. The diameter of the upper opening 49 that defines the circular access opening through the cap can be from about 7 mm to 13 mm, such as from 5 mm to 11 mm. The circular access opening can be a diameter that is from 10% to 90%, such as from 25% to 75% of the inner diameter of the upper injection end 65 of the barrel (shown as inner diameter 63 in FIG. 3). The thickness 97 of the cap can be the same or about the same (±0.2 mm) in all locations of the cap.

As one option in the present invention, the cap presses the septum against at least the inner sidewall of the barrel. Thus, the septum, as a result, can be compressed, to some extent, against at least part of or a portion of the inner sidewall of the injection site end of the barrel. The amount of compression can be such that the septum is compressed in a uniaxial or biaxial direction by less than 3%, such as 0.01% to 1% (comparing a non-compressed septum to a compress septum with respect to total surface area) or amounts below or above this range. This compression can be a uniaxial compression or a biaxial compression. The compression can result in the septum being squeezed or compressed so as to exhibit only from 90% to 99.99% (e.g. 98% to 99.99%) of its original, non-compressed height and/or non-compressed diameter. The interlocking nature of the complementary ridge and notch of the cap and barrel (due to molding design of each part), respectively, can in part secure (e.g. lock) the cap and barrel together with the compressed septum compressed there between. This compression can contribute or provide a ‘self-sealing’ feature to the septum once an injection needle or cannula has been removed from the septum. This compression can impart a pre-load to the septum and improve the sealing ability of the septum after a needle or cannula is removed from the septum. As indicated, any ridge or notch or similar design present in any of the barrel, septum, and/or cap is created from the mold design and formed during the molding process.

The cap 7 can comprise, consist essentially of, consist of, or include, at least one thermoplastic material. The thermoplastic material described herein and used to form the barrel can be used to form the cap and those details are incorporated herein by reference. The material to form the cap can be the same or different thermoplastic polymer(s) used to form the barrel. The material used to form the cap can contain, in addition to the thermoplastic material, one or more additives from the list of additives that can be present to form the barrel and again, those details are incorporated herein by reference.

Generally, the material used to form the cap contains at least 90 wt % thermoplastic polymer(s), such as from 90 wt % to 100 wt %, or from 90 wt % to 99 wt %, or from 90 wt % to 95 wt %, based on the total weight of the material used to form the cap. The material used to form the cap can be the same material or a different material from the material used to form the barrel.

Once all of the parts of the injection port are formed (e.g., the cap, the septum, and the barrel are injection molded), in a preferred design, except for the designed fluid path of the inner bore of the barrel, there is an absence or de minimis amount of any trapped air spaces between the cap and the septum, and/or between the septum and the barrel, and/or between the cap and the barrel, and preferably no air spaces or trapped air at any of these three areas of contact. Preferably, in the injection port of the present invention, there is no exposed air pockets amongst the cap, septum, and barrel. Preferably, any amount of trapped air would be to such a low amount that post-sterilization of the injection port after being manufactured would not be needed.

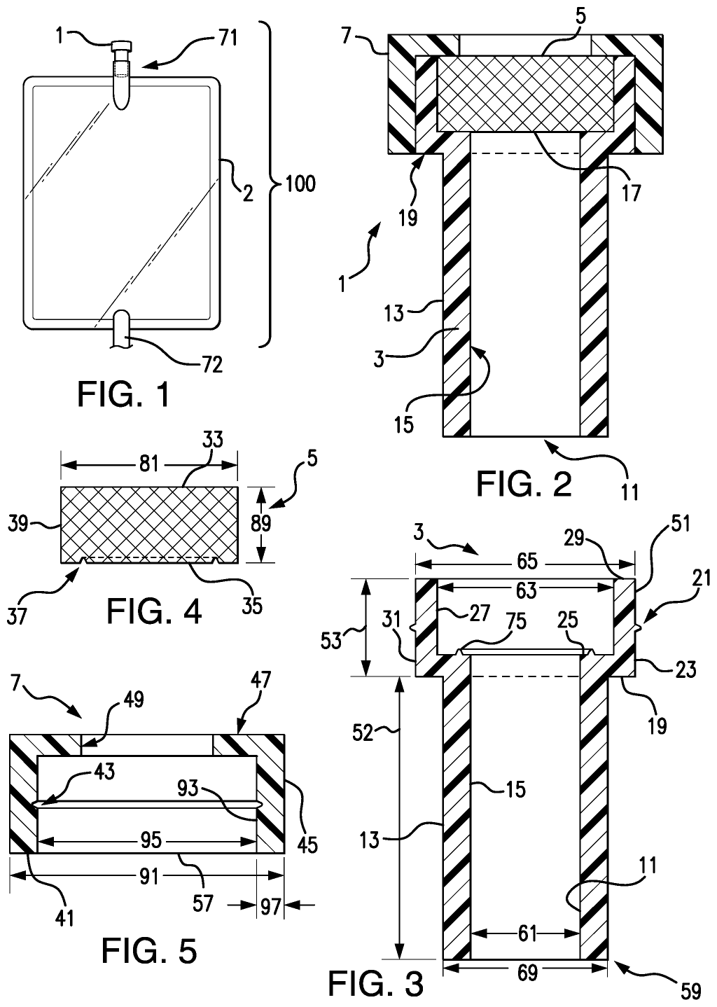

The present invention further relates to an assembly 100. The assembly comprises, consists essentially of, consists of, or includes the injection port 1 of the present invention and a container 2, such as a fluid container or a medical fluid container, wherein the container (e.g. medical fluid container) has an interior and the tubular bore of the barrel is in fluid communication with the interior of the container, such as by tube 71 in FIG. 1. The barrel can be secured to the container (e.g. via tube 71) in any manner such as by pressure fitting, heat sealing, a Luer connector, or by any other connection means and/or techniques. The container 2 can contain two or more tubes where one of the tubes 71 is connected to the injection port 1 of the present invention, and the other one or tubes 72 are means to connect the fluid container to a dispensing device or to a gravity feed device (not shown). The injection port is generally used to dispense a drug or other additive to the fluid container and the other tube or conduit is used to dispense the fluid from the fluid container.

As indicated previously, the fluid in the container can be a dialysis fluid, an intravenous fluid, a wash fluid, sterile water, sterile saline, ringers lactate, dextrose (such as 5% dextrose), or other IV fluids, or any other liquid that is located in an interior chamber of the container.

The present invention further relates to a method of making the injection port of the present invention. The method can comprise, consist essentially of, consist of, or include utilizing a three-shot injection molding process.

In such a process, during a first shot of the three-shot injection molding process, the barrel is formed. Then, during a second shot of the three-shot injection molding process, the septum is formed. And then, during the third shot of the three-shot injection molding process, the cap is formed.

The three-shot injection molding process can use a commercially available injection molding machine, such as, but not limited to, injection molding machines from Arburg GmbH, Milacron, Husky, Engle, Arburg, Krauss-Maffei, Milacron, Nissei and the like. The injection molding machine can be an electric, hydraulic, or servo type injection molding machine.

As an option, the third shot can apply a compression on the septum that presses the septum against an inner shoulder of the barrel. The details of this option are described herein and apply equally here.

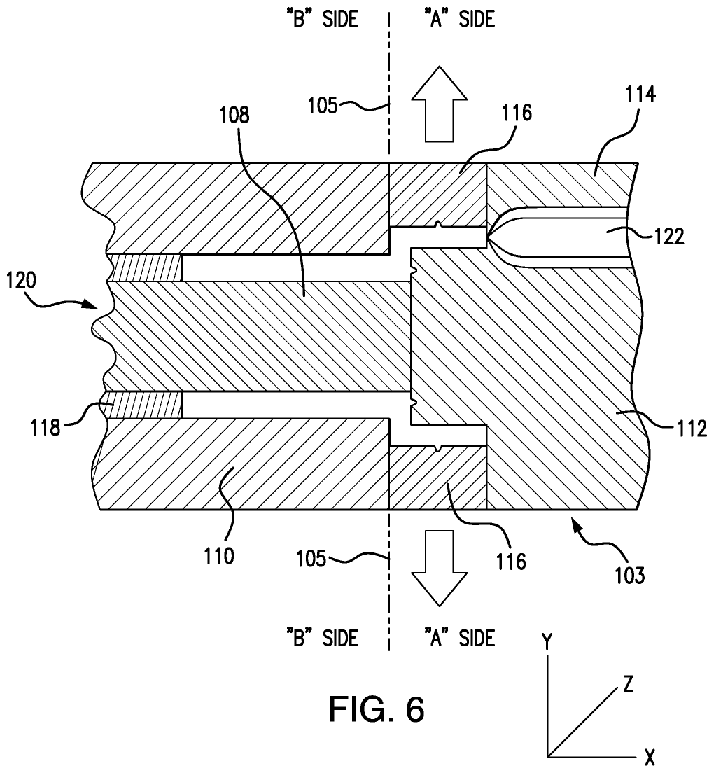

After the first shot of the three-shot injection molding process, the barrel is cooled to a temperature that permits the barrel to retain its molded barrel shape before the second shot of the three-shot injection molding process is carried out. The barrel can be cooled by a liquid cooling jacket, or liquid cooling channels, or a combination thereof, within the mold. Further, the mold can have a thermocouple that measures the temperature of the mold and/or the object within the mold so that the mold is not opened until a temperature is reached that reflects at least partial or complete solidification of the molded piece. This temperature monitoring can be used during all three injection and cooling steps of the molding process, that is, during the injection and cooling of each of the three shots. In the molds used and depicted in FIGS. 6-8, it is understood that liquid cooling channels (not shown) are present for this purpose.

After the first shot is injected and cooled to form the barrel, the second shot is then injected and cooled to form the septum. Once the second shot is injected, thermal bonding between the surface of the inner surfaces of the barrel and the contacting surfaces of the septum can occur. Once the second shot is injected, cooling can be controlled so that a portion of the barrel becomes semi-solid or melts. When some melting is caused or occurs, this can result in a portion of the barrel material combining with a portion of the septum material to form an interface of the two materials. If this occurs, a thermally bonded interface between the barrel and the septum can be formed. This interface can be relatively thin (e.g. less than 10 microns or less than 5 microns or less than 1 micron in thickness), or can occur only at the surfaces of the two components. As an option, after the second shot of the three-shot injection molding process, is injected, the septum can be cooled to a temperature that enables the septum to retain its molded septum shape before the third shot of the three-shot injection molding process is injected.

After injection and cooling of the first and second shots, the third shot can be injected to form the cap. Once the third shot is injected, thermal bonding between the surface of the cap and the contacting surfaces of the septum and/or barrel can occur. Once the third shot is made, a portion of the barrel and/or septum can become a semi-solid or melt, and this can result in a portion of the barrel material and/or septum material combining with a portion of the cap material to form one or more thermally-bonded interfaces between the components. If this occurs, the interface(s) can be relatively thin (e.g. less than 10 microns or less than 5 microns or less than 1 micron in thickness), or can occur only at the surfaces of the contacting components.

The first, second, and third shots can follow the sequence of injection steps shown in FIGS. 6, 7, and 8, respectively, which exemplify one type of mold assembly that can be utilized. It is to be understood that other molding steps or processes can be used. The molds utilized in FIGS. 6-8 have two sides, an “A” side and a “B” side. The “A” side can be considered the injection side and the “B” side can be considered the ejection side. A parting line (P/L) in each of FIGS. 6, 7, and 8 show the separation line 105 of “A” and “B” sides. The parting line is where the A side of the mold and the B side of the mold mate in each molding step. The “B” side 110 in FIGS. 6-8 is same in each of FIGS. 6, 7, and 8. The “A” side in each of FIGS. 6, 7, and 8 is different from each other and contain the cavity geometry to mold the next sequential piece of the assembly. The B side can be mounted or located on a rotary platen so that this B side of the mold can rotate to the location for the first shot, and then rotate to the location for the second shot, and then rotate to the location of the third shot.

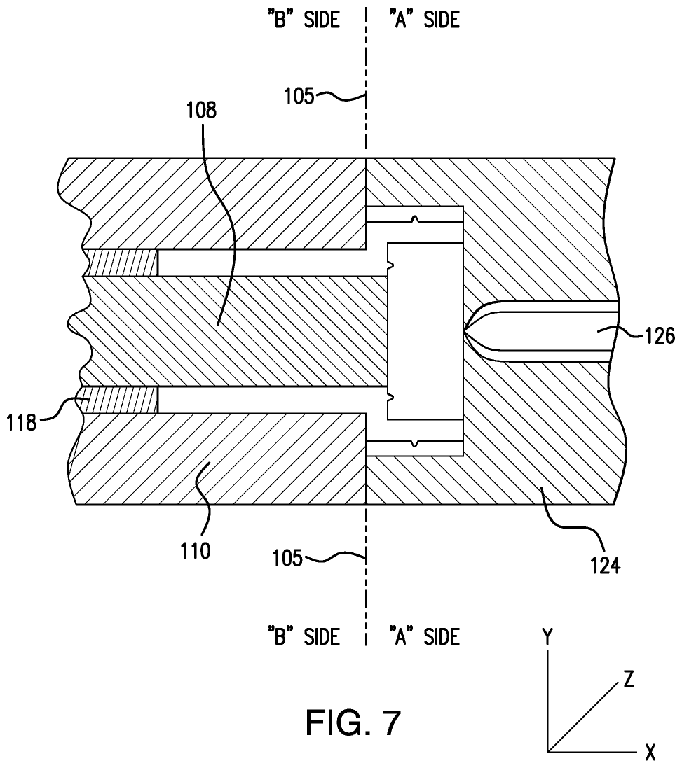

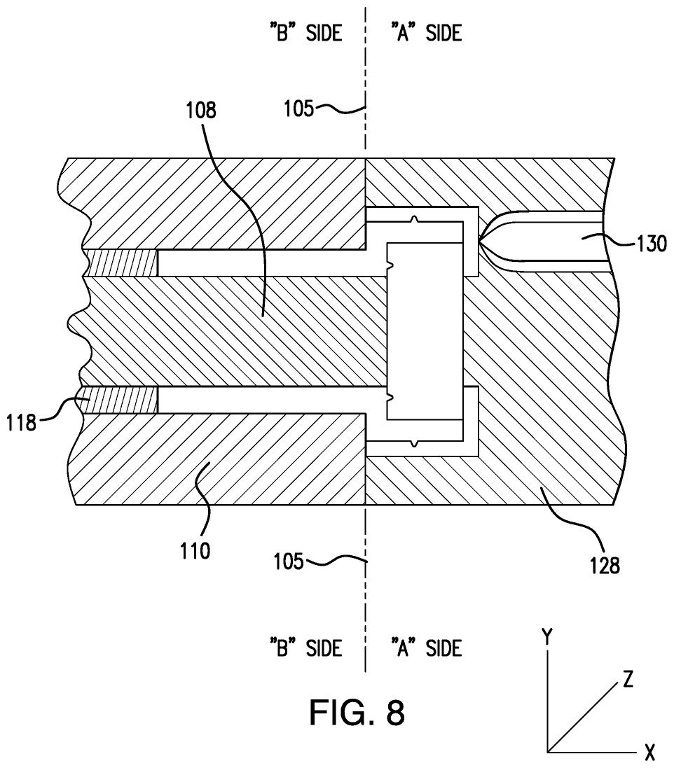

At the first shot, the A side 112 for the first shot is mated with the B side as shown in FIG. 6. In FIG. 6, the A side comprises an A side mold cavity 114 and at least two A side cavity slides 116. As also shown in FIG. 6, an ejector sleeve 118 is located in the core 120 adjacent the core pin 108. As shown in FIG. 6, the geometry of the barrel is shown and this is filled with the material that forms the barrel and this material can be injected into the mold using a gate tip or hot runner gate tip 122. Upon solidification of the barrel piece, the A side mold cavity opens. The mold opens by removing the A side of the mold which involves sliding out the side cavity slides 116 first and then removing the A side mold cavity 114. The A side cavity slides 116 and the A side mold cavity 114 remain at the first station so as to be placed on the next B side mold cavity that arrives. Once done, the B side with the barrel molded piece rotates to the next station (FIG. 7) and upon arriving at the next station, the A side mold cavity 124 is mated with the B side mold cavity 110 as shown in FIG. 7. The B side mold cavity 124 has the geometry to form the septum piece. Once A side mold cavity 124 is in place, the material used to form the septum is injected into the mold by way of hot runner gate tip 126. Afterwards, upon solidification of the piece, the A side mold cavity 124 opens and the B side, now with the molded barrel and septum, rotates to the third station for the third shot, as shown in FIG. 8. The A side mold cavity 124 remains at the second station so as to be placed on the next B side mold cavity that arrives. In FIG. 8, the A side mold cavity 128 for the third shot (which has the geometry of the cap) is mated to the B side mold cavity 110 and then material for forming the cap is injected into hot runner gate tip 130. Upon solidification of the cap piece, the A side mold cavity 128 opens, and the ejector sleeve 118 can move in the X direction to eject the completely formed injection port of the present invention. The A side mold cavity 128 remains at the third station so as to be placed on the next B side mold cavity that arrives.

As an option, a system can be provided for operating a molding machine to carry out the three-shot molding process. The system can include a controller, for example, a computer processor with memory, programmed to carry out a set of instructions, for example, to carry out a sequence of process steps. Molten polymer and molten elastomer pumps or injection units can be part of the system and can be controlled by the controller. A cooling system pump or multiple pumps can be part of the system and can be controlled by the controller to pump coolant through cooling channels and jackets to effect a cooling of the molten polymer or molten elastomer after injection. A heating subsystem can be part of the system and can include a heater, controlled by the controller, to independently melt polymer and elastomer and maintain molten materials at certain temperatures. Drivers including motors and robotic arms can be used to move the various components of the system and drive the pumps. The controller, drivers, and robotic arms can be configured to open and close mold parts, pump or inject molten polymer and molten elastomer through different injection ports, extend and retract push rods to control positioning of plungers, control temperatures and pumping or injection times and pressures, and time the process steps to sequentially carry out the three-step molding process. Hydraulic actuators, pneumatic actuators, or electronic stepper motor actuators can be used to cause movement of system components, and the hydraulic actuators can be controlled by the controller. Exemplary components that can be incorporated into the system can include those described, for example, in U.S. Pat. Nos. 3,951,388; 3,999,923; 4,019,845; 4,032,277; 4,070,139; 4,410,478; 4,592,712; 4,592,714; 4,599,063; and 4,605,367, each of which is incorporated herein in its entirety by reference.

The present invention includes the following aspects/embodiments/features in any order and/or in any combination:

1. The present invention relates to an injection port for connecting with a medical fluid container, said injection port comprising a) a barrel having a tubular bore for fluid communication with an interior of the medical fluid container, and an injection site end configured to hold a septum; b) a septum in contact with the injection site end of the barrel and providing a sealing partition to the injection site end of the barrel, the septum being configured to be resealable after being pierced by an injection needle or cannula; and c) a cap having an annular shape, circumscribing the septum, holding at least partly the septum to the barrel at the injection site end, and having a central opening enabling access to the septum, said cap further circumscribing the injection site end of the barrel, wherein said cap, in part, secures the septum in place, and wherein the barrel, the septum, and the cap are sequentially formed via a three-shot injection molding process.
2. The injection port of any preceding or following embodiment/feature/aspect, wherein said barrel comprises a notch at the injection site end, the notch circumscribing an outside surface of the barrel at the injection site end, and wherein said cap has a complimentary barb configured to engage the notch and secure said cap on said barrel.
3. The injection port of any preceding or following embodiment/feature/aspect, wherein said barrel has a base, said base has an inner diameter, said injection site end has an inner diameter and an inner sidewall, and the inner diameter of the base is smaller than the inner diameter of the injection site end of the barrel.
4. The injection port of any preceding or following embodiment/feature/aspect, wherein said septum is located within the injection site end of the barrel such that the outer sidewall of the septum is in contact with the inner sidewall of the injection site end of the barrel.
5. The injection port of any preceding or following embodiment/feature/aspect, wherein the injection site end of the barrel defines an inner shoulder that circumscribes the barrel, the septum has an inner axial surface, an outer axial surface, and an outer sidewall, and a portion of the inner axial surface of the septum rests against the inner shoulder.
6. The injection port of any preceding or following embodiment/feature/aspect, wherein the cap presses the septum against at least the inner sidewall of the barrel.
7. The injection port of any preceding or following embodiment/feature/aspect, wherein the cap, septum, and barrel are injection molded such that there is an absence of any trapped air spaces between the cap and the septum, between the septum and the barrel, and between the cap and the barrel.
8. The injection port of any preceding or following embodiment/feature/aspect, wherein each of the barrel and the cap comprises a thermoplastic material and the septum comprises an elastomeric material that is different from said thermoplastic material.
9. The injection port of any preceding or following embodiment/feature/aspect, wherein the barrel and the cap comprise the same thermoplastic material and the septum comprises an elastomeric material that is different from said thermoplastic material utilized for the barrel, cap or both.
10. The injection port of any preceding or following embodiment/feature/aspect, wherein the medical fluid container has an interior and the tubular bore of the barrel is in fluid communication with the interior of the medical fluid container.
11. The injection port of any preceding or following embodiment/feature/aspect, wherein the medical fluid container comprises dialysis fluid in an interior chamber of the medical fluid container.
12. A method of making the injection port of any preceding or following embodiment/feature/aspect, wherein said method comprising a three-shot injection molding process, wherein, during a first shot of the three-shot injection molding process, the barrel is formed, during a second shot of the three-shot injection molding process, the septum is formed, and during the third shot of the three-shot injection molding process, the cap is formed.
13. The method of any preceding or following embodiment/feature/aspect, wherein the third shot applies compression on the septum and presses the septum against an inner shoulder of the barrel.
14. The method of any preceding or following embodiment/feature/aspect, wherein the barrel further comprises a connecting end, and the method further comprises heat-sealing the connecting end to a medical fluid container such that the tubular bore of the barrel is in fluid communication with an interior of the medical fluid container.
15. The method of any preceding or following embodiment/feature/aspect, wherein the barrel further comprises a connecting end, and the method further comprises connecting the connecting end to a medical fluid container such that the tubular bore of the barrel is in fluid communication with an interior of the medical fluid container.
16. The method of any preceding or following embodiment/feature/aspect, wherein, after the first shot of the three-shot injection molding process, the barrel is cooled to a temperature that permits the barrel to retain its molded barrel shape before the second shot of the three-shot injection molding process is carried out.
17. The method of any preceding or following embodiment/feature/aspect, wherein, after the second shot of the three-shot injection molding process, the septum is cooled to a temperature that permits the septum to retain its molded septum shape before the third shot of the three-shot injection molding process is carried out.
18. The method of any preceding or following embodiment/feature/aspect, wherein said medical fluid container is an infusion fluid container.

The present invention can include any combination of these various features or embodiments above and/or below as set forth in sentences and/or paragraphs. Any combination of disclosed features herein is considered part of the present invention and no limitation is intended with respect to combinable features.

Applicants specifically incorporate the entire contents of all cited references in this disclosure. Further, when an amount, concentration, or other value or parameter is given as either a range, a preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. An injection port for connecting with a medical fluid container, said injection port comprising a) a barrel having a tubular bore for fluid communication with an interior of the medical fluid container, and an injection site end configured to hold a septum; b) a septum in contact with the injection site end of the barrel and providing a sealing partition to the injection site end of the barrel, the septum being configured to be resealable after being pierced by an injection needle or cannula; and c) a cap having an annular shape, circumscribing the septum, holding at least partly the septum to the barrel at the injection site end, and having a central opening enabling access to the septum, said cap further circumscribing the injection site end of the barrel, wherein said cap, in part, secures the septum in place, and wherein the barrel, the septum, and the cap are sequentially formed via a three-shot injection molding process.

2. The injection port of claim 1, wherein said barrel comprises a notch at the injection site end, the notch circumscribing an outside surface of the barrel at the injection site end, and wherein said cap has a complimentary barb configured to engage the notch and secure said cap on said barrel.

3. The injection port of claim 1, wherein said barrel has a base, said base has an inner diameter, said injection site end has an inner diameter and an inner sidewall, and the inner diameter of the base is smaller than the inner diameter of the injection site end of the barrel.

4. The injection port of claim 3, wherein said septum has an outer diameter and an outer sidewall, and wherein said septum is located within the injection site end of the barrel such that the outer sidewall of the septum is in contact with the inner sidewall of the injection site end of the barrel.

5. The injection port of claim 1, wherein the injection site end of the barrel defines an inner shoulder that circumscribes the barrel, the septum has an inner axial surface, an outer axial surface, and an outer sidewall, and a portion of the inner axial surface of the septum rests against the inner shoulder.

6. The injection port of claim 5, wherein the cap presses the septum against at least the inner sidewall of the barrel.

7. The injection port of claim 1, wherein the cap, septum, and barrel are injection molded such that there is an absence of any trapped air spaces between the cap and the septum, between the septum and the barrel, and between the cap and the barrel.

8. The injection port of claim 1, wherein each of the barrel and the cap comprises a thermoplastic material and the septum comprises an elastomeric material that is different from said thermoplastic material.

9. The injection port of claim 1, wherein the barrel and the cap comprise the same thermoplastic material and the septum comprises an elastomeric material that is different from said thermoplastic material utilized for the barrel, cap or both.

10. An assembly comprising the injection port of claim 1 and a medical fluid container, wherein the medical fluid container has an interior and the tubular bore of the barrel is in fluid communication with the interior of the medical fluid container.

11. The assembly of claim 10, wherein the medical fluid container comprises dialysis fluid in an interior chamber of the medical fluid container.

12. A method of making the injection port of claim 1, said method comprising a three-shot injection molding process, wherein, during a first shot of the three-shot injection molding process, the barrel is formed, during a second shot of the three-shot injection molding process, the septum is formed, and during the third shot of the three-shot injection molding process, the cap is formed.

13. The method of claim 12, wherein the third shot applies compression on the septum and presses the septum against an inner shoulder of the barrel.

14. The method of claim 12, wherein the barrel further comprises a connecting end, and the method further comprises heat-sealing the connecting end to a medical fluid container such that the tubular bore of the barrel is in fluid communication with an interior of the medical fluid container.

15. The method of claim 12, wherein the barrel further comprises a connecting end, and the method further comprises connecting the connecting end to a medical fluid container such that the tubular bore of the barrel is in fluid communication with an interior of the medical fluid container.

16. The method of claim 12, wherein, after the first shot of the three-shot injection molding process, the barrel is cooled to a temperature that permits the barrel to retain its molded barrel shape before the second shot of the three-shot injection molding process is carried out.

17. The method of claim 16, wherein, after the second shot of the three-shot injection molding process, the septum is cooled to a temperature that permits the septum to retain its molded septum shape before the third shot of the three-shot injection molding process is carried out.

18. The method of claim 12, wherein said medical fluid container is an infusion fluid container.
